(19) United States
(12) Patent Application Publication
(10) Pub. No.: US 2025/0152761 A1
(43) Pub. Date: May 15, 2025

(54) MULTIFUNCTIONAL ULTRASONIC CLEANING DEVICE

(71) Applicant: Jing Li, Cili (CN)

(72) Inventor: Jing Li, Cili (CN)

(21) Appl. No.: 19/028,102

(22) Filed: Jan. 17, 2025

(30) Foreign Application Priority Data
Jan. 10, 2025 (CN) 202520058600.7

(51) Int. Cl. A61L 2/025; A61L 2/10

(52) U.S. Cl. CPC A61L 2/025; A61L 2/10; A61L 2202/121; A61L 2202/122

(57) ABSTRACT

A multifunctional ultrasonic cleaning device is provided, including a device body; the device body is provided with a cleaning cylinder and a drying cylinder; a flip cover is covered at a position, located an opening of the drying cylinder, on the flip cover; a drying mechanism is arranged at a projection region of the opening of the drying cylinder; the drying mechanism includes an air-driving fan blade group and an air guide member; a lower end of the air guide member extends into the drying cylinder; the air guide member is provided with an air blocking enclosure wall around a circumferential periphery of an air gathering duct; and a junction between the flip cover and the device body is formed into an air outlet gap.

### TECHNICAL FIELD

The present disclosure relates to the technical field of cleaning devices, and in particular, to a multifunctional ultrasonic cleaning device.

### BACKGROUND

An ultrasonic cleaning device mainly uses an energy converter to convert a high-frequency oscillation signal transmitted by an ultrasonic generator into high-frequency mechanical oscillation by an energy converter and propagates the high-frequency mechanical oscillation into a cleaning agent. Ultrasonic waves are radiated forwards alternately densely and sparsely in a cleaning solution, to cause the solution to flow and generate tens of thousands of tiny bubbles. These tiny bubbles vibrate under the action of a sound field, and are formed and grow in a negative pressure region of longitudinal propagation of the ultrasonic waves, and are quickly closed in a positive pressure region. In this “cavitation” effect process, the closure of the bubbles can generate an instantaneous high pressure of over 1000 Pa. The continuously generated high pressures continuously impact a surface of an object, like a series of small “explosion”, causing dirt on the surface of the object and in gaps to be quickly peeled off, thus achieving a purpose of surface cleaning.

After the ultrasonic cleaning, a small amount of water marks and a cleaning agent are inevitably attached to an item and food after the item and the food are ultrasonically cleaned. Therefore, before use of the cleaned item or food, a user often uses a dry product such as tissue to wipe it. However, in this process, the item or the food is easily subjected to secondary pollution. This mode hardly meets an ideal hygiene need.

### SUMMARY

The present disclosure aims to provide a multifunctional ultrasonic cleaning device. A cleaning cylinder and a drying cylinder are integrally arranged on the device. When a need for treatment after cleaning is met, the possibility of secondary pollution to an item in a transferring process is minimized. A quick drying effect can be achieved in the drying cylinder through optimization on an air path and heating treatment. Meanwhile, the device further has a sterilization function, meets a higher hygiene need of a user, and solves the problems in the prior art.

To achieve the above objectives, the present disclosure provides the following technical solution: A multifunctional ultrasonic cleaning device includes a device body; the device body is provided with a cleaning cylinder and a drying cylinder; a flip cover is covered at a position, located at an opening of the drying cylinder, on the flip cover; a drying mechanism is arranged at a projection region of the opening of the drying cylinder; the drying mechanism includes an air-driving fan blade group and an air guide member; when the flip cover is in covered connection to the drying cylinder, a lower end of the air guide member extends into the drying cylinder; a height of the air guide member from a position leveled with the opening of the drying cylinder to a bottom surface of the air guide member is set to H, H≥12 mm; the air guide member includes an air gathering duct extending in a lengthwise direction of the air guide member; a second air inlet is formed by the air gathering duct in the lower end of the air guide member; a first air inlet for communicating inside to outside of the drying cylinder is provided in the drying mechanism; the air-driving fan blade group guides air at the first air inlet to flow through the air gathering duct and then be blown into the drying cylinder from the second air inlet; the air guide member is provided with an air blocking enclosure wall around a circumferential periphery of the air gathering duct; a thickness distance between the air blocking enclosure wall and the air gathering duct is set to W, W≥8 mm; and a junction between the flip cover and the device body is formed into an air outlet gap.

Preferably, when the flip cover is in covered connection to the drying cylinder, a gap D communicated to the air outlet gap exists between the air blocking enclosure wall and an inner wall surface of the drying cylinder, D≥3 mm.

Preferably, the drying mechanism includes a heating element arranged at the second air inlet.

Preferably, a drainage frame is detachably placed inside the drying cylinder; an isolation pad is arranged between the drainage frame and a bottom surface of the drying cylinder; a first magnetic suction member is fixedly arranged on the isolation pad; a second magnetic suction member is arranged on an outer side of the bottom surface of the drying cylinder; the isolation pad is detachably connected by magnetic forces of the first magnetic suction member and the second magnetic suction member.

Preferably, a first accommodating chamber is formed between the air blocking enclosure wall and the air gathering duct; a lamp panel is arranged in the first accommodating chamber; the lamp panel is encapsulated with ultraviolet sterilization LEDs; LED through holes are formed in the lower end of the air guide member; the ultraviolet sterilization LEDs are arranged corresponding to the LED through holes; and light emitted by the ultraviolet sterilization LEDs irradiates the interior of the drying cylinder through the LED through holes.

Preferably, the first air inlet is arranged in a manner of penetrating through upper and lower surfaces of the flip cover.

Preferably, one side edge of the flip cover is rotatably connected to the device body through a rotating mechanism; the flip cover includes a flip cover panel and a flip cover bottom plate in covered connection to the flip cover panel; and a transparent observation window is provided in a position, located at an opening of the cleaning cylinder, of the flip cover.

Preferably, an opening of the cleaning cylinder is in water seal connection to a housing of the device body through a sealing gasket; an ultrasonic generator is arranged on an outer side of a bottom surface of the cleaning cylinder; and the cleaning cylinder is fixedly connected to the device body through a cleaning cylinder bracket.

Preferably, the device body includes an upper device housing and a lower device housing in covered connection to the upper device housing; a second accommodating chamber is formed between the upper device housing and the lower device housing; a circuit board assembly is mounted in the second accommodating chamber; the circuit board assembly is electrically connected with touch buttons, a display screen, and a USB jack; the touch buttons and the display screen are arranged on the flip cover; and the USB jack is provided in a through hole provided in the upper device housing in advance and extends out of the through hole.

Compared with the prior art, the present disclosure has the beneficial effects below:

The device is integrally provided with the cleaning cylinder and the drying cylinder, so that when a need for treatment after cleaning is met, the possibility of secondary pollution to an item in a transferring process is minimized. A quick drying effect can be achieved in the drying cylinder through optimization on an air path and heating treatment. Meanwhile, the device further has a sterilization function. The device has multiple functions, is practical, and meets a higher hygiene need of a user.

### BRIEF DESCRIPTION OF THE DRAWINGS

FIG. 1 is a structural exploded view I of the present disclosure;

FIG. 2 is a structural exploded view II of the present disclosure;

FIG. 3 is a reference diagram I of a use state of the present disclosure;

FIG. 4 is a reference diagram II of a use state of the present disclosure;

FIG. 5 is a schematic structural diagram of an air guide member according to the present disclosure;

FIG. 6 is a radial projection view of an air guide member according to the present disclosure; and

FIG. 7 is an axial projection view of an air guide member according to the present disclosure.

In the drawings: 1: flip cover; 11: flip cover panel; 12: flip cover bottom plate; 121: air outlet gap; 122: first air inlet; 123: transparent observation window; 13: air-driving fan blade group; 14: lamp panel; 141: ultraviolet sterilization LED; 15: air guide member; 151: air gathering duct; 152: air blocking enclosure wall; 153: LED through hole; 154: second air inlet; 16: heating element; 2: device body; 21: cleaning cylinder; 211: sealing gasket; 212: cleaning cylinder bracket; 213: ultraviolet generator; 22: drying cylinder; 221: drainage frame; 222: isolation pad; 223: first magnetic suction member; 224: second magnetic suction member; 23: upper device housing; 24: circuit board assembly; 241: touch button; 242: display screen; 243: USB jack; and 25: lower device housing.

### DETAILED DESCRIPTION OF THE EMBODIMENTS

The technical solutions in the embodiments of the present disclosure will be clearly and completely described below with reference to the drawings in the embodiments of the present disclosure, and it is obvious that the described embodiments are only a part of the embodiments of the present disclosure but not all of them. All other embodiments obtained by a person of ordinary skill in the art based on the embodiments of present disclosure without making creative efforts shall fall within the protection scope of present disclosure.

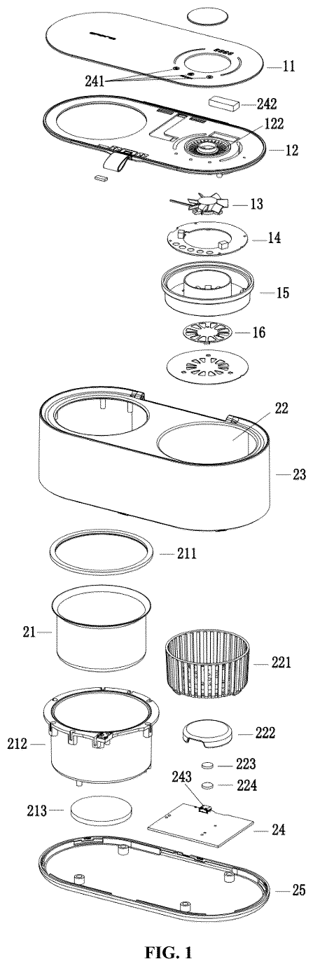

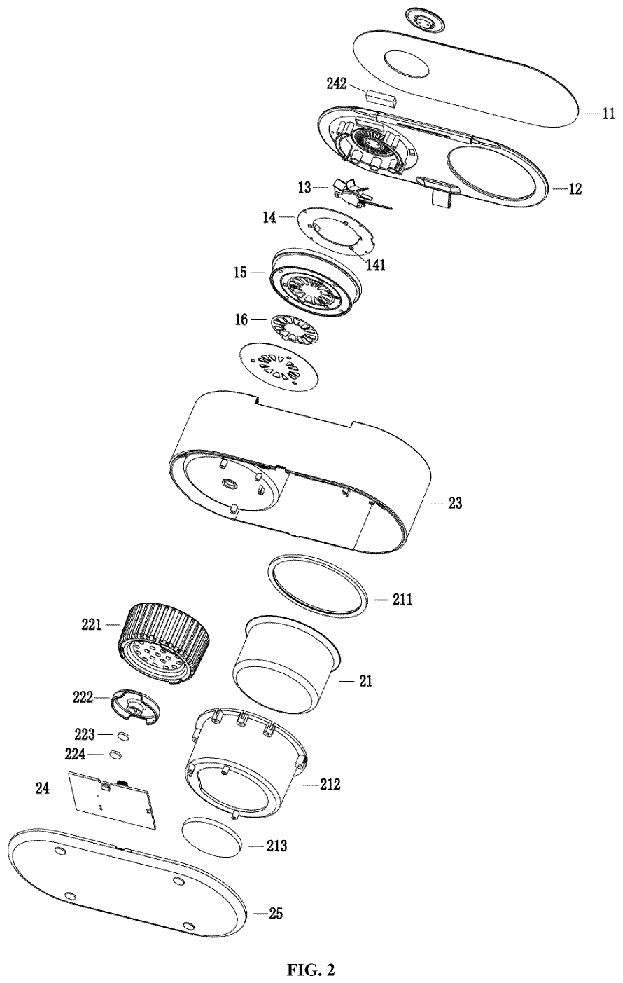

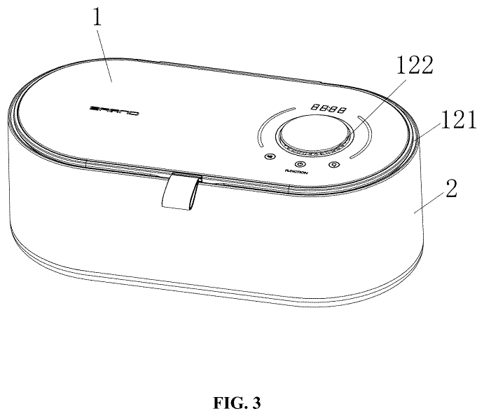

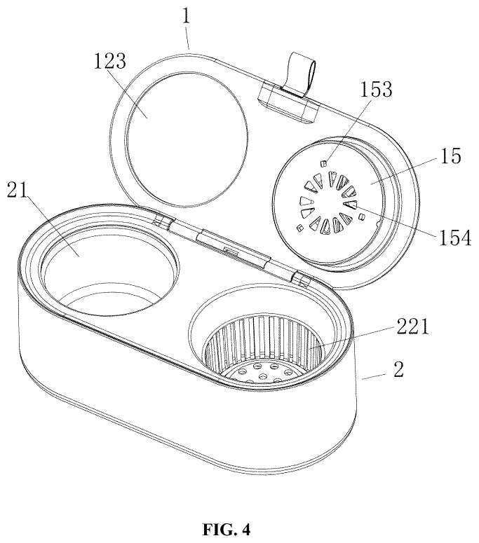

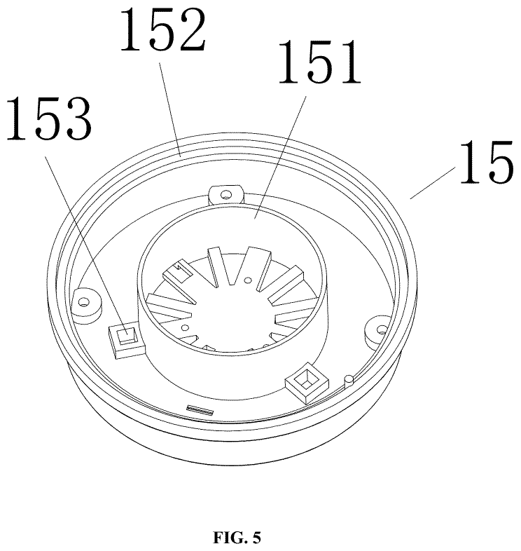

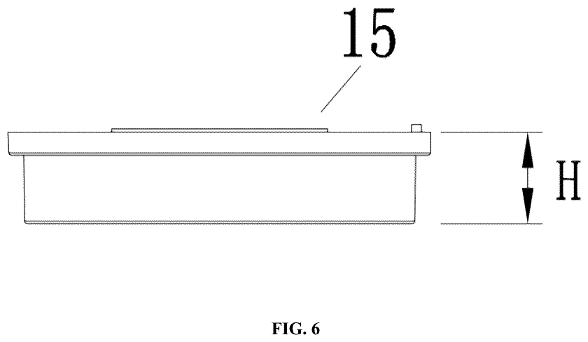

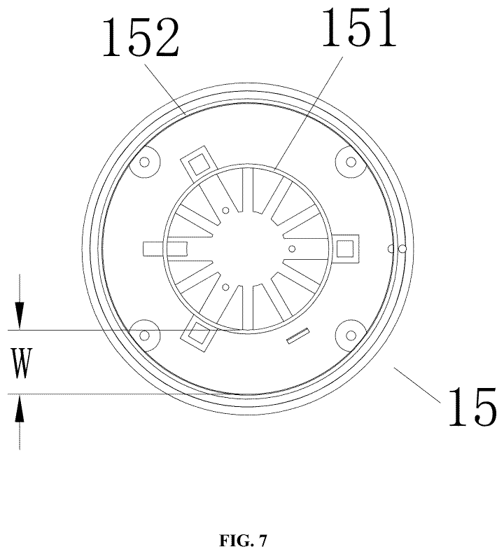

Referring to FIG. 1 to FIG. 7, a multifunctional ultrasonic cleaning device is provided, including a device body 2; the device body 2 is provided with a cleaning cylinder 21 and a drying cylinder 22; a flip cover 1 is covered at an opening of the drying cylinder 22; one side edge of the flip cover 1 is rotatably connected to the device body 2 through a rotating mechanism; the flip cover 1 includes a flip cover panel 11 and a flip cover bottom plate 12 in covered connection to the flip cover panel 11; a transparent observation window 123 is provided in a position, located at an opening of the cleaning cylinder 21, of the flip cover 1; a drying mechanism is arranged at a position, located at a projection region of the opening of the drying cylinder 22, on the flip cover 1; the drying mechanism includes an air-driving fan blade group 13 and an air guide member 15; when the flip cover 1 is in covered connection to the drying cylinder 22, a lower end of the air guide member 15 extends into the drying cylinder 22; and a height of the air guide member 15 from a position leveled with the opening of the drying cylinder 22 to a bottom surface of the air guide member 15 is set to H, H≥12 mm. The air guide member 15 includes an air gathering duct 151 extending in a lengthwise direction of the air guide member 15; a second air inlet 154 is formed by the air gathering duct 151 in the lower end of the air guide member 15; a first air inlet 122 for communicating inside to outside of the drying cylinder 22 is provided in the drying mechanism; the first air inlet 122 is arranged in a manner of penetrating through upper and lower surfaces of the flip cover 1; the air-driving fan blade group 13 guides air at the first air inlet 122 to flow through the air gathering duct 151 and then be blown into the drying cylinder 22 from the second air inlet 154; the air gathering duct 151 can achieve an effect of gathering air for pressurization; the air guide member 15 is provided with an air blocking enclosure wall 152 around a circumferential periphery of the air gathering duct 151; and a thickness distance between the air blocking enclosure wall 152 and the air gathering duct 151 is set to W, W≥8 mm. The air blocking enclosure wall 152 is configured to block a returned air flow to cause the returned air flow to reversely flow out of an air outlet gap, thus avoiding addition of a turbulence effect due to collision between the returned air flow and an inlet air flow as much as possible. A junction between the flip cover 1 and the device body 2 is formed into an air outlet gap 121. When the flip cover 1 is in covered connection to the drying cylinder 22, a gap D communicated to the air outlet gap 121 exists between the air blocking enclosure wall 152 and an inner wall surface of the drying cylinder 22, D≥3 mm. Structural features, i.e. H, W, and D, are configured to define a direction of an air flow inside the drying cylinder, so that a trajectory of an air path is clearer, which effectively avoids the phenomenon that the drying effect is affected by an unclear air path and mixing of dry air and wet air due to the turbulence caused by lack of the air guide member 15 for guidance in an air path in a traditional drying device, and effectively improves the drying effect of an air flow on an item.

The drying mechanism includes a heating element 16 arranged at the second air inlet 154. The heating element 16 heats the air flow of the drying cylinder 22, to improve the drying efficiency.

A drainage frame 221 is detachably placed inside the drying cylinder 22; an isolation pad 222 is arranged between the drainage frame 221 and a bottom surface of the drying cylinder 22; a first magnetic suction member 223 is fixedly arranged on the isolation pad 222; a second magnetic suction member 224 is arranged on an outer side of the bottom surface of the drying cylinder 22; the isolation pad 222 is detachably connected by magnetic forces of the first magnetic suction member 223 and the second magnetic suction member 224.

A first accommodating chamber is formed between the air blocking enclosure wall 152 and the air gathering duct 151; a lamp panel 14 is arranged in the first accommodating chamber; the lamp panel 14 is encapsulated with ultraviolet sterilization LEDs 141; LED through holes 153 are formed in the lower end of the air guide member 15; the ultraviolet sterilization LEDs 141 are arranged corresponding to the LED through holes 153; and light emitted by the ultraviolet sterilization LEDs 141 irradiates the interior of the drying cylinder 22 through the LED through holes 153. The ultraviolet sterilization LEDs 141 are configured to sterilize an item in the drying cylinder and can achieve an effect of sterilization and elimination of a residual cleaning agent.

The device body 2 includes an upper device housing 23 and a lower device housing 25 in covered connection to the upper device housing 23; an opening of the cleaning cylinder 21 is in water seal connection to a housing of the device body 2 through a sealing gasket 211; an ultrasonic generator 213 is arranged on an outer side of a bottom surface of the cleaning cylinder 21; the cleaning cylinder 21 is fixedly connected to the device body 2 through a cleaning cylinder bracket 212; a second accommodating chamber is formed between the upper device housing 21 and the lower device housing 25; a circuit board assembly 24 is mounted in the second accommodating chamber; the circuit board assembly 24 is electrically connected with touch buttons 241, a display screen 242, and a USB jack 243; the touch buttons 241 and the display screen 242 are arranged on the flip cover 1; and the USB jack 243 is provided in a through hole provided in the upper device housing 23 in advance and extends out of the through hole. The air-driving fan blade group 13, the lamp panel 14, the heating element 16, and the ultrasonic generator 213 are all electrically connected to the circuit board assembly 24. The touch buttons 241 are configured to control and adjust working states of the above working components. The display screen 242 is configured to display working parameters of the above working components. The USB jack 243 supplies working power to the device when connected to an external power supply through a charging cable.

In summary: According to the present disclosure, the device is integrally provided with the cleaning cylinder 21 and the drying cylinder 22, so that when a need for treatment after cleaning is met, the possibility of secondary pollution to an item in a transferring process is minimized. A quick drying effect can be achieved in the drying cylinder 22 through optimization on an air path and heating treatment. Meanwhile, the device further has a sterilization function. The device has multiple functions, is practical, and meets a higher hygiene need of a user.

Although the embodiments of the present disclosure have been shown and described, it can be understood by those of ordinary skill in the art that various changes, modifications, substitutions, and variations can be made to these embodiments without departing from the principle and spirit of the present disclosure. The scope of the present disclosure is defined by the accompanying claims and their equivalents.

What is claimed is:

1. A multifunctional ultrasonic cleaning device, comprising a device body (2), wherein the device body (2) is provided with a cleaning cylinder (21) and a drying cylinder (22); a flip cover (1) is covered at an opening of the drying cylinder (22); a drying mechanism is arranged at a position, located at a projection region of the opening of the drying cylinder (22), on the flip cover (1); the drying mechanism comprises an air-driving fan blade group (13) and an air guide member (15); when the flip cover (1) is in covered connection to the drying cylinder (22), a lower end of the air guide member (15) extends into the drying cylinder (22); a height of the air guide member (15) from a position leveled with the opening of the drying cylinder (22) to a bottom surface of the air guide member (15) is set to H, H≥12 mm; the air guide member (15) comprises an air gathering duct (151) extending in a lengthwise direction of the air guide member (15); a second air inlet (154) is formed by the air gathering duct (151) in the lower end of the air guide member (15); a first air inlet (122) for communicating inside to outside of the drying cylinder (22) is provided in the drying mechanism; the air-driving fan blade group (13) guides air at the first air inlet (122) to flow through the air gathering duct (151) and then be blown into the drying cylinder (22) from the second air inlet (154); the air guide member (15) is provided with an air blocking enclosure wall (152) around a circumferential periphery of the air gathering duct (151); a thickness distance between the air blocking enclosure wall (152) and the air gathering duct (151) is set to W, W≥8 mm; and a junction between the flip cover (1) and the device body (2) is formed into an air outlet gap (121).

2. The multifunctional ultrasonic cleaning device according to claim 1, wherein when the flip cover (1) is in covered connection to the drying cylinder (22), a gap D communicated to the air outlet gap (121) exists between the air blocking enclosure wall (152) and an inner wall surface of the drying cylinder (22), D≥3 mm.

3. The multifunctional ultrasonic cleaning device according to claim 1, wherein the drying mechanism comprises a heating element (16) arranged at the second air inlet (154).

4. The multifunctional ultrasonic cleaning device according to claim 1, wherein a drainage frame (221) is detachably placed inside the drying cylinder (22); an isolation pad (222) is arranged between the drainage frame (221) and a bottom surface of the drying cylinder (22); a first magnetic suction member (223) is fixedly arranged on the isolation pad (222); a second magnetic suction member (224) is arranged on an outer side of the bottom surface of the drying cylinder (22); the isolation pad (222) is detachably connected by magnetic forces of the first magnetic suction member (223) and the second magnetic suction member (224).

5. The multifunctional ultrasonic cleaning device according to claim 1, wherein a first accommodating chamber is formed between the air blocking enclosure wall (152) and the air gathering duct (151); a lamp panel (14) is arranged in the first accommodating chamber; the lamp panel (14) is encapsulated with ultraviolet sterilization LEDs (141); LED through holes (153) are formed in the lower end of the air guide member (15); the ultraviolet sterilization LEDs (141) are arranged corresponding to the LED through holes (153); and light emitted by the ultraviolet sterilization LEDs (141) irradiates the interior of the drying cylinder (22) through the LED through holes (153).

6. The multifunctional ultrasonic cleaning device according to claim 1, wherein the first air inlet (122) is arranged in a manner of penetrating through upper and lower surfaces of the flip cover (1).

7. The multifunctional ultrasonic cleaning device according to claim 1, wherein one side edge of the flip cover (1) is rotatably connected to the device body (2) through a rotating mechanism; the flip cover (1) comprises a flip cover panel (11) and a flip cover bottom plate (12) in covered connection to the flip cover panel (11); and a transparent observation window (123) is provided in a position, located at an opening of the cleaning cylinder (21), of the flip cover (1).

8. The multifunctional ultrasonic cleaning device according to claim 1, wherein an opening of the cleaning cylinder (21) is in water seal connection to a housing of the device body (2) through a sealing gasket (211); an ultrasonic generator (213) is arranged on an outer side of a bottom surface of the cleaning cylinder (21); and the cleaning cylinder (21) is fixedly connected to the device body (2) through a cleaning cylinder bracket (212).

9. The multifunctional ultrasonic cleaning device according to claim 1, wherein the device body (2) comprises an upper device housing (23) and a lower device housing (25) in covered connection to the upper device housing (23); a second accommodating chamber is formed between the upper device housing (21) and the lower device housing (25); a circuit board assembly (24) is mounted in the second accommodating chamber; the circuit board assembly (24) is electrically connected with touch buttons (241), a display screen (242), and a USB jack (243); the touch buttons (241) and the display screen (242) are arranged on the flip cover (1); and the USB jack (243) is provided in a through hole provided in the upper device housing (23) in advance and extends out of the through hole.
